(19) United States
(12) Patent Application Publication
(10) Pub. No.: US 2012/0077250 A1
(43) Pub. Date: Mar. 29, 2012

(54) METHOD FOR CULTURING PHOTOSYNTHETIC MICROORGANISMS ON MICROBIAL CELLULOSE

(75) Inventor: Barbara R. Evans, Oak Ridge, TN (US)

(73) Assignee: UT-BATTELLE, LLC, Oak Ridge, TN (US)

(21) Appl. No.: 12/888,703

(22) Filed: Sep. 23, 2010

(51) Int. Cl. C12N 1/20; C12N 1/12; C12N 1/00

(52) U.S. Cl. 435/252.1; 435/243; 435/257.2; 435/257.3; 435/257.5; 435/257.6

(57) ABSTRACT

This invention provides methods for growing and maintaining photosynthetic microorganisms such as algae and cyanobacteria. More specifically, the invention provides methods of cultivation of photosynthetic microorganisms on bacterial cellulose.

### STATEMENT REGARDING FEDERALLY SPONSORED RESEARCH OR DEVELOPMENT

This invention was made with government support under Prime Contract No. DE-AC05-00OR22725 awarded by the U.S. Department of Energy. The government has certain rights in the invention.

### FIELD OF THE INVENTION

This invention pertains to growth and cultivation of photosynthetic microorganisms such as algae and cyanobacteria. More specifically, the invention relates to cultivation of photosynthetic microorganisms by utilizing microbial cellulose as matrix.

### BACKGROUND OF THE INVENTION

Algae are a large group of photosynthetic organisms. Green algae represent a large subgroup from which higher plants emerged. In contrast, the organisms that were previously referred to as “blue-green algae” are currently classified as cyanobacteria. However, algae and cyanobacteria have certain properties in common, including photosynthesis.

There have been attempts to apply the properties of algae and cyanobacteria to industrial and medical problems. For example, algae and cyanobacteria produce oxygen during photosynthesis, and U.S. Pat. No. 5,614,378 issued Mar. 25, 1997 to Yang et al. discloses a photobioreactor system for oxygen production for a closed ecological life support system. Additionally, some algae and cyanobacteria produce hydrogen gas under anaerobic conditions, and U.S. Pat. No. 7,176,005 B2, issued Feb. 13, 2007 to Melis et al. discloses sustained hydrogen production by culturing genetically modified algae. Furthermore, some algae and cyanobacteria can fix nitrogen, and U.S. Pat. No. 5,093,262 issued Mar. 3, 1992 to Kimura discloses a method for producing organic fertilizer involving green algae.

Several species of acetic acid bacteria are known to produce a skin or pellicule of pure cellulose at the air-liquid interface. For example, these bacteria are cultivated in the Philippines on coconut milk and sucrose to form cellulose pellicules used in the food product nata de coco.

European Patent Application 0 243 151 A2, published Oct. 28, 1987 naming Yamanaka et al. as inventors (“Yamanaka et al.”) discloses an “ordinary nutrient culture medium” and microbially-produced cellulose derived from cultures of a cellulose-producing microbe in this “ordinary nutrient culture medium.” Yamanaka et al. also discloses microbially-produced cellulose modified by i) physically or chemically bonding an animal cell adhesive protein to the cellulose; or ii) substituting hydrogen atoms of at least some hydroxyl groups of the cellulose with a positively or negatively charged organic group. Yamanaka et al. discloses that the number of animal cells adhering to unmodified cellulose is much fewer than the number of animal cells adhering to a plastic petri dish or with a cellulose-producing microbe, and allowing the culture medium to stand still or gently stirring the culture medium under aeration.

### SUMMARY OF THE INVENTION

The present invention relates to growth and cultivation of photosynthetic microorganisms.

In one embodiment, the invention provides a method for culturing a photosynthetic microorganism by growing the photosynthetic microorganism on sufficiently transparent bacterial cellulose.

The present method can be applied to photosynthetic microorganisms selected from the group consisting of a green alga, a cyanobacterium, a purple photosynthetic bacterium, or a diatom. Examples of green algae include members of a genus selected from the group consisting of Chlamydomonas, Chlorella, Parachlorella, Pseudochlorella, Bracteococcus, Prototheca, Scenedesmus, and Serenastrus. In a specific embodiment, the green alga is a member of the species Chlamydomonas reinhardtii.

The bacterial cellulose suitable for use in cultivating a photosynthetic microorganism can be produced from a cellulose-producing bacterium, for example, a bacterium of a genus selected from the group consisting of Gluconacetobacter, Acetobacter, and Gluconobacter. In certain embodiments, the bacterial cellulose is the product of a bacterium of a species selected from the group consisting of Gluconacetobacter europaeus, Gluconacetobacter hansenii, and Gluconacetobacter xylinus. In a specific embodiment, the bacterial cellulose is the product of a bacterium of the Gluconacetobacter hansenii species.

In accordance with the present invention, cellulose producing microbes are cultured in a synthetic minimal medium, in order to produce relatively transparent bacterial cellulose suitable for growing a photosynthetic microorganism.

The bacterial cellulose isolated from the culture medium can be used directly for culturing photosynthetic microorganisms, but preferably, the bacterial cellulose is treated to remove bacterial proteins and media components prior to use for culturing photosynthetic microorganisms.

In some embodiments, the bacterial cellulose is complexed with an appropriate auxiliary material for the purposes of reinforcement, expanding surface area, and easy handling, for example. In one embodiment, bacterial cellulose forms composites with terrestrial plant-derived cellulose.

In a further aspect, the invention provides a bacterial cellulose assembly comprising a photosynthetic microorganism immobilized on the surface of bacterial cellulose.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 documents the growth of green algae on microbial cellulose. Cultures of green algae (Chlamydomonas reinhardtii) in minimal salts medium for algae (see Table 1) were inoculated into culture dishes. The cultures were incubated at 23° C. for 16 days, during which the cultures were illuminated with white fluorescent light of a luminous intensity of 12 μE/m²/s. Panel A is an image of a culture of microbial cellulose in minimal salts medium for algae without C. reinhardtii; panel B is an image of minimal salts medium for algae without microbial cellulose alone; panel C is an image of a culture of microbial cellulose and C. reinhardtii in minimal salts medium for algae; and panel D is an image of a culture of C. reinhardtii in minimal salts medium for algae.

FIG. 2 documents the growth of green algae on sterile plugging cotton. Cultures of C. reinhardtii in minimal acetate medium were inoculated into flasks. The flasks were incubated at 23° C. for ten days, during which the cultures were illuminated with white fluorescent light of a luminous intensity of 3 μE/m²/s. The left-hand flask is a culture of C. reinhardtii in minimal acetate medium while the right-hand flask is a culture of C. reinhardtii and sterile plugging cotton in minimal acetate medium.

FIG. 3 documents the absorbance of hydrated microbial cellulose and microbial cellulose dried with a vacuum gel drier (Hoefer) as described in U.S. Pat. No. 6,986,963 B2, issued Jan. 17, 2006 to Evans et al. and Evans, B. R., O'Neill, H. M., Malyvanh, V. P., Lee, I. Woodward, J. “Palladium-bacterial cellulose membranes for fuel cells”, (2003) Biosens. Bioelectron. 18(7), 917-923. The upper line represents the absorbance of hydrated microbial cellulose; the middle line represents the absorbance of dried microbial cellulose; and the lower line represents the absorbance of an empty spectrophotometer cuvette.

### DETAILED DESCRIPTION OF THE INVENTION

This invention is directed to methods for cultivating photosynthetic microorganisms. More specifically, the invention is based on the utilization of microbial cellulose as a support for immobilization, growth, and maintenance of photosynthetic microorganisms such as green algae and cyanobacteria.

Characteristics of Suitable Bacterial Cellulose

The terms “microbial cellulose”, “microbially produced cellulose” and “bacterial cellulose” are used interchangeably herein, and refer to cellulose produced by a microbe.

In accordance with the present invention, bacterial cellulose has been produced in a form that is porous, highly hydrated and relatively transparent to visible light to permit immobilization, growth, and maintenance of photosynthetic microorganisms.

By “highly hydrated”, it is meant that the bacterial cellulose is in the form of a gel or pellicules containing a large amount of water, i.e., having a water content of at least 90%, 95%, 98%, or even 99% or greater (v/v).

The term “pellicule”, as used herein, refers to the type of bacterial cellulose that takes the form of film or layer.

By “relatively transparent” or “sufficiently transparent” is meant that the bacterial cellulose permits sufficient transmission of light in the photosynthetically active region (PAR) to permit growth of photosynthetic microorganisms. Transparency can be determined, for example, by illuminating the bacterial cellulose of interest with light in the PAR and measuring the absorbance by using a spectrometer. The PAR generally refers to light having a wavelength in the range of 390 nm to 750 nm, or preferably in the range of 400 nm to 700 nm, in which photosynthetic pigments such as chlorophyll absorb. As used herein, a material can be considered to be sufficiently transparent if its absorbance of light in the PAR is not more than 0.6. In specific embodiments, the bacterial cellulose has an absorbance of light at a wavelength within the PAR of not more than 0.4, or preferably not more than 0.3.

Bacterial cellulose in the form of a gel or pellicules has a structure characterized by ribbon-shaped microfibrils entangled with one another and pores having a mean pore size of at least about 0.05 microns and not more than 500 microns, and in some embodiments at least about 0.1 microns, or from about 0.1 microns to about 15 microns. Bacterial cellulose produced in accordance with the present invention has pores with a mean pore size of several microns, e.g., 3-10 microns, or 5-10 microns in some embodiments.

Bacterial cellulose having the above-described features offers a number of advantages for cultivation of photosynthetic microorganisms as compared to cellulosic materials derived from plants, including its sponge-like hydrated surface, its transmission of visible light required for photosynthesis, and its biocompatibility with microorganisms and living tissues.

Production of Bacterial Cellulose

Bacterial cellulose having the above-described features can be produced using any of cellulose-producing microbes, including microbes belonging to the genera Gluconacetobacter (which includes species formerly classified as Acetobacter), Acetobacter, and Gluconoabacter. In some embodiments, bacterial species of the genus Gluconacetobacter are used for producing cellulose, for example, Gluconacetobacter europaeus, Gluconacetobacter hansenii, and Gluconacetobacter xylinus. In a specific embodiment, Gluconacetobacter hansenii (ATCC 10821) is used for the production of bacterial cellulose.

In accordance with the present invention, cellulose producing microbes are cultured in a synthetic minimal medium, rather than a conventional rich medium, in order to produce relatively transparent bacterial cellulose suitable for growing a photosynthetic microorganism.

By “synthetic medium” it is meant herein a chemically defined culture medium consisting of a reproducible solution with defined and known inorganic and/or organic compound, as compared to a conventional “rich medium”, which has complex ingredients, such as yeast extract, and contains a mixture of multiple chemical species in unknown proportions.

By “synthetic minimal medium” it is meant herein a synthetic medium that contains minimal nutrients required for bacterial growth, including a defined carbon source, a nitrogen source, inorganic salts, and optionally trace elements and vitamins. Generally speaking, to achieve effective production of bacterial cellulose, glucose, cellobiose, fructose, sucrose, glycerol, sorbitol, or mannitol, or a combination thereof, or one or more of the aforementioned carbon sources supplemented with ethanol and/or acetate, can be used as the carbon source, ammonium salts such as ammonium chloride, ammonium sulfate, ammonium phosphate can be used as the nitrogen source, and additional inorganic salts which can be selected from sodium, potassium, magnesium, calcium, phosphate, or a combination thereof, for example. Vitamins such as niacinamide, thiamine and calcium pantothenate, or a source thereof such as yeast extract, yeast autolysate, extracts derived from plants or algae including apple juice, grape juice, or tea infusions prepared from China tea (Camellia sinensis), are also included in the minimal medium.

In some embodiments, the synthetic minimal medium used for culturing cellulose-producing microbes include glucose, cellobiose, fructose, mannitol or a combination thereof as the carbon source, ammonium chloride as the nitrogen source, citric acid, sodium dihydrogen phosphate, potassium chloride, magnesium sulfate heptahydrate, supplemented with niacinamide, thiamine, and calcium pantothenate.

Typically, the carbon source is used at a concentration of 1% (w/v) to 5% (w/v), preferably about 2% (w/v), and the ammonium salt is used at a concentration of about 0.1% (w/v).

In a specific embodiment, the synthetic minimal medium has the following formulation: 2% glucose, cellobiose, fructose, mannitol or a combination thereof, 0.1% ammonium chloride, 0.115% citric acid, 0.33% sodium dihydrogen phosphate, 0.01% potassium chloride, and 0.025% magnesium sulfate heptahydrate, supplemented with 10 mg/l niacinamide, 10 mg/l thiamine, and 10 mg/l calcium pantothenate.

To produce cellulose, a synthetic minimal medium is inoculated with a preculture of a cellulose-producing microbe. The culture is allowed to stand still, i.e., under static conditions without stirring, at a temperature in the range of 20° C. to 40° C., preferably at about 23° C., or at about 30° C., for about 7-21 days. The cellulose can be harvested from the surface layer of the culture medium.

In some embodiments, the harvested cellulose is used directly for cultivating a photosynthetic microbe. That is, the bacterial cellulose gel isolated from the culture medium, which may contain certain impurities (such as sugar, salts, culture medium and microbial cells) can be used directly as a matrix for cultivating a photosynthetic microbe.

In other embodiments, the bacterial cellulose isolated from the culture medium is treated to remove bacterial proteins and media components and to kill bacterial cells. For example, the harvested bacterial cellulose can heated to 90° C. in distilled water, washing with 1% sodium hydroxide solution, and finally extensive washing with distilled water. Afterwards the cellulose can be sterilized by steam sterilization in distilled water in an autoclave.

In accordance with the present invention, while the intact bacterial cellulose harvested from a static bacterial culture is used without being pulped, macerated, or modified by covalent attachment of chemical groups, in some embodiments, the bacterial cellulose can be complexed with an appropriate auxiliary material for the purposes of reinforcement, expanding surface area, and easy handling, for example. Suitable auxiliary materials for use include, for example, fabrics composed of natural cellulosic fibers (such as cotton and linen) or man-made fibers (such as regenerated celluloses and polyesters), paper sheets, polyvinyl alcohol, crystalline celluloses, and water-soluble or polar solvent-soluble materials or hydrophilic gel-forming materials such as agar, dextran, polyacrylamide, polyvinylpyrrolidone, alginic acid salts, chitin, hyaluronic acid, curdlan, polyacrylic acid salts, pullulan, carrageenan, glucomannan, cellulose derivatives and polyethylene glycol.

To make bacterial cellulose that is complexed with a desirable auxiliary material, the material can be incorporated in the culture medium and the microbially-produced cellulose is formed on the surface or in the interior of said substance. Alternatively, the bacterial cellulose in the form of a gel obtained from the culture medium can be impregnated or backed with the auxiliary material of interest.

In another embodiment, the cellulose-producing microbes are grown on terrestrial plant-derived cellulose to produce cellulose composites consisting of bacterial cellulose layers on terrestrial plant-derived cellulose. Such composites can be used to provide greater surface area and amenability to assembly in bioreactors.

Cultivation of Photosynthetic Microorganisms Using Bacterial Cellulose

The culturing method based on utilization of bacterial cellulose having the above-described features can be applied to grow and maintain photosynthetic microorganisms, including for example, a green alga, a cyanobacterium, a purple photosynthetic bacterium, or a diatom. Examples of green algae contemplated by the invention include Chlamydomonas (e.g., Chlamydomonas reinhardtii), Chlorella (e.g., Chlorella ellipsoidea, Chlorella kessleri, Chlorella luteoviridis, Chlorella minutissima, Chlorella ovalis, Chlorella protothecoides, Chlorella pyrenoidosa, Chlorella saccharophila, Chlorella sorokiniana, and Chlorella vulgaris), Parachlorella (e.g., Parachlorella kessleri), Pseudochlorella (e.g., Pseudochlorella aquatic), Bracteococcus (Bracteococcus minor and Bracteococcus medionucleatus), Prototheca (e.g., Prototheca moriformis), Scenedesmus (e.g., Scenedesmus obligus and Scenedesmus auadricauda), and Serenastrus (e.g., Serenastrum capricornutum). Examples of a purple photosynthetic bacterium include members of the genus Rhodobacter (e.g., Rhodobacter sphaeroides). An example of a diatom is Thalassiosira pseudonana.

Bacterial cellulose in the form of a gel or pellicles can be placed in any suitable culture device, such as culture dishes, plates or flasks, and can be soaked in minimal salts medium suitable for culturing a photosynthetic microbe before inoculation with the photosynthetic microbe. Afterwards, the photosynthetic microbe is seeded onto the bacterial cellulose. This can be done by, e.g., adding a diluted pre-culture of the photosynthetic microbe to the culture dish in which the relatively transparent bacterial cellulose is placed, and adding a minimal salts medium commonly used for culturing the photosynthetic microbe. The culture is provided with white fluorescent light and incubated at an appropriate temperature. The growth of the photosynthetic microbe can be periodically evaluated by removing the medium from the culture dish, and measuring the absorbance of the medium at 680 nm, the characteristic absorption maximum of chlorophyll, in a spectrophotometer; or simply examining the culture dish by eye.

In one embodiment, the minimal salts medium for algae is set forth in Table 1 (Composition of Minimal Salts Medium for Algae Modified by Increased Phosphate and Magnesium Concentrations from Sueoka, N. (1960) Proc. Natl. Acad. Sci. USA 46, 83-91):.

| | | | FINAL | |
|---|---|---|---|---|
| COMPONENT | FORMULA | COMPOUND | CONC. | |
| Bierjinck's Salts | NH₄Cl | Ammonium chloride | 8.0 | mM |
| | CaCl₂•2H₂O | Calcium chloride dihydrate | 350 | μM |
| | MgSO₄•2H₂O | Magnesium sulfate dihydrate | 410 | μM |
| Phosphate buffer | K₂HPO₄ | Potassium phosphate dibasic | 4.1 | mM |
| | KH₂PO₄ | Potassium phosphate monobasic | 3.0 | mM |
| Trace Elements | ZnSO₄•7H₂O | Zinc sulfate heptahydrate | 77 | μM |
| | H₃BO₃ | Boric acid | 184 | μM |
| | MnCl₂•4H₂O | Manganese (II) chloride | 26 | μM |
| | | tetrahydrate | | |
| | CoCl₂•6H₂O | Cobalt (II) chloride hexahydrate | 6.8 | μM |
| | CuSO₄•5H₂O | Copper (II) sulfate pentahydrate | 6.3 | μM |
| | (NH₄)₆Mo₇O₂₄•H₂O | Ammonium molybdate (VI) | 0.89 | μM |
| | | tetrahydrate | | |
| | FeSO₄•7H₂O | Iron(II) sulfate hetpahydrate | 18 | μM |
| | EDTA, free acid | Ethylenediaminetetraacetate | 171 | μM |

In another embodiment, minimal acetate medium is minimal salts medium for algae containing 2 g/L sodium acetate.

As identified in accordance with the present invention, photosynthetic bacteria can colonize and grow on bacterial cellulose, and become immobilized or absorbed to bacterial cellulose, yet remain on the surface of the bacterial cellulose matrix, allowing easy removal of spent medium and faster growth.

Additional Utilities

The bacterial cellulose membranes assembled for algal cultivation can be used for growth of algae for bioproducts including starch and lipids, for which the algae can be harvested, and for production of small biofuel molecules particularly hydrogen and ethanol. When utilized to grow algae for starch to be used for bioethanol production by yeast fermentation, the entire bacterial cellulose assembly containing the algae can be hydrolyzed to glucose and other sugars by digestion with cellulase and amylase enzymes followed by yeast fermentation. The algae containing starch and the bacterial cellulose cultivation support can also be fermented directly to ethanol or butanol by fermentation with certain Clostridial bacteria that are known to hydrolyze cellulose and starch.

### EXAMPLE

Bacterial cellulose pellicules with 10 cm diameter were prepared by cultivation of the bacterium Gluconacetobacter hansenii (ATCC 10821) in 10-cm culture dishes under static conditions with a synthetic minimal medium consisting of 2% sugar or other carbon source, 0.1% ammonium chloride, 0.115% citric acid, 0.33% sodium dihydrogen phosphate, 0.01% potassium chloride, 0.025% magnesium sulfate heptahydrate, supplemented with 100 mg/l niacinamide, 100 mg/l thiamine, and 100 mg/l calcium pantothenate, in which the carbon source used is glucose, cellobiose, fructose, or mannitol

The cellulose was purified by heating to 90° C. in distilled water following harvest, washing with 1% sodium hydroxide solution to remove bacterial proteins and media components, and finally extensive washing with distilled water. The cellulose was sterilized by steam sterilization in distilled water in an autoclave. Cellulose pellicles were placed in each of two 10-cm sterile culture dishes. The pellicles were soaked in minimal salts medium for algae for 1 hour. The excess medium was then decanted. Two empty sterile 10-cm dishes were used as controls.

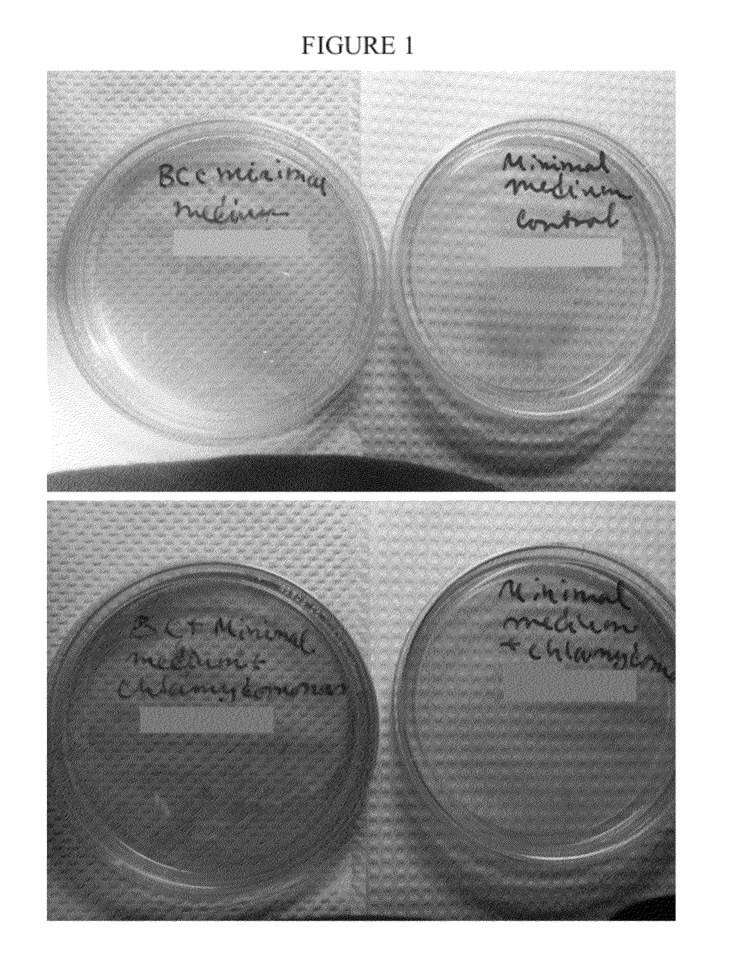

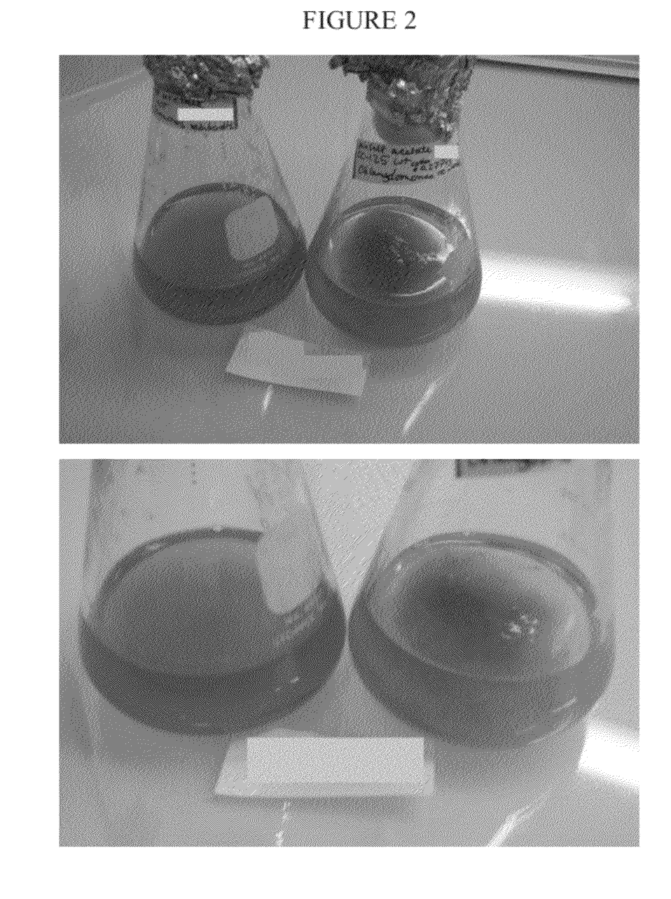

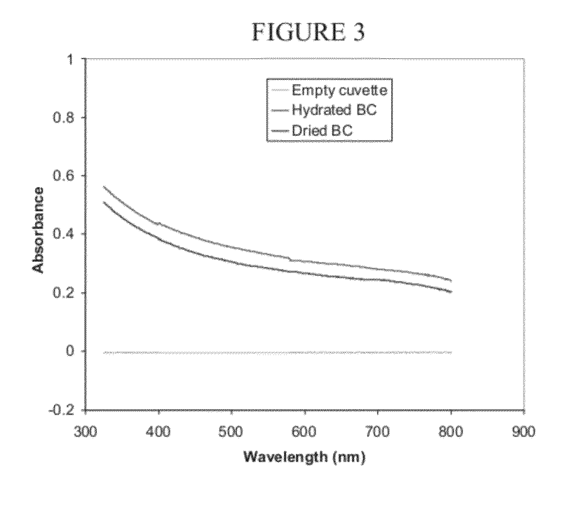

A culture of green algae, Chlamydomonas reinhardtii C137⁺, CC-125, a wild-type strain commonly used in research, was diluted 1/100 (0.4 ml algal culture to 40 ml minimal salts medium for algae). The algae had been grown for 20 days at 10 μE/m²/s white fluorescent light. Ten ml of the diluted algal culture was added to each culture dish. The culture dishes were labeled as minimal salts medium for algae control, Chlamydomonas control, minimal salts medium for algae with cellulose, and Chlamydomonas with cellulose. The dishes were incubated at 23° C. and 12 μE/m²/s white fluorescent light. After 16 days, the medium was removed from each of the dishes and the algal growth estimated by measuring the absorbance of the medium at 680 nm, the characteristic absorption maximum of chlorophyll, in a spectrophotometer. Twenty milliliters of fresh minimal salts medium for algae were then added to each dish, and incubation was continued under the same conditions. The medium was again removed after 23 days and the algae estimated as before. The experiment was terminated after 57 days. Growth of the algae appeared to be better sustained by the bacterial cellulose than in the dish without cellulose, as shown in FIGS. 1-3. No contamination or deterioration was observed in the control dishes without algae.

What is claimed is:

1. A method for culturing a photosynthetic microorganism, comprising culturing the photosynthetic microorganism on sufficiently transparent bacterial cellulose.

2. The method according to claim 1, wherein the photosynthetic microorganism is selected from the group consisting of a green alga, a cyanobacterium, a purple photosynthetic bacterium, or a diatom.

3. The method according to claim 2, wherein the green alga is a member of a genus selected from the group consisting of Chlamydomonas, Chlorella, Parachlorella, Pseudochlorella, Bracteococcus, Prototheca, Scenedesmus, and Serenastrus.

4. The method according to claim 3, wherein the green alga is a member of the species Chlamydomonas reinhardtii.

5. The method according to claim 3, wherein the green alga is a member of the species selected from the group consisting of Chlorella ellipsoidea, Chlorella kessleri, Chlorella luteoviridis, Chlorella minutissima, Chlorella ovalis, Chlorella protothecoides, Chlorella pyrenoidosa, Chlorella saccharophila, Chlorella sorokiniana, Chlorella vulgaris, Parachlorella kessleri, Pseudochlorella aquatic, Bracteococcus minor, Bracteococcus medionucleatus, Prototheca moriformis, Scenedesmus obliges, Scenedesmus auadricauda, and Serenastrum capricornutum.

6. The method according to claim 2, wherein the purple photosynthetic bacterium is a member of the genus Rhodobacter.

7. The method according to claim 2, wherein the diatom is a member of the species Thalassiosira pseudonana.

8. The method according to claim 1, wherein the bacterial cellulose is a product of a bacterium of a genus selected from the group consisting of Agrobacterium, Gluconacetobacter, Pseudomonas, Rhizobium, and Sarcina.

9. The method according to claim 8, wherein the bacterial cellulose is the product of a bacterium of a species selected from the group consisting of Gluconacetobacter europaeus, Gluconacetobacter hansenii, and Gluconacetobacter xylinus.

10. The method of claim 1, wherein the bacterial cellulose is produced from a cellulose-producing bacterium by culturing said bacterium in a synthetic minimal medium under static conditions.

11. The method of claim 10, wherein said synthetic minimal medium comprises 2% w/v of a carbon source selected from the group consisting of glucose, cellobiose, fructose, mannitol, sucrose, glycerol, sorbitol, or a combination, a nitrogen source, a source of vitamins, and inorganic salts.

12. The method of claim 1, wherein the bacterial cellulose is complexed with an auxiliary material.

13. The method of claim 1, wherein the culturing of the photosynthetic microorganism comprises culturing the photosynthetic microorganism in a minimal medium on the bacterial cellulose, and illuminating the culture with white fluorescent light.

14. An assembly comprising bacterial cellulose in the form of a gel or pellicules, and a photosynthetic bacterium immobilized on the surface of said gel or pellicules.

15. The assembly of claim 14, wherein the photosynthetic microorganism is selected from the group consisting of a green alga, a cyanobacterium, a purple photosynthetic bacterium, or a diatom.

16. The assembly of claim 15, wherein the green alga is a member of a genus selected from the group consisting of Chlamydomonas, Chlorella, Parachlorella, Pseudochlorella, Bracteococcus, Prototheca, Scenedesmus, and Serenastrus.

17. The assembly of claim 16, wherein the green alga is a member of the species Chlamydomonas reinhardtii.
